(19) United States
(12) Patent Application Publication
(10) Pub. No.: US 2022/0042045 A1
(43) Pub. Date: Feb. 10, 2022

(54) EXPRESSION CASSETTES FOR GENE THERAPY VECTORS

(71) Applicants: GENETHON, EVRY (FR); INSTITUT NATIONAL DE LA SANTE ET DE LA RECHERCHE MEDICALE, PARIS (FR); UNIVERSITE D'EVRY VAL D'ESSONNE, EVRY (FR)

(72) Inventors: ANA BUJ BELLO, PARIS (FR); MARTINA MARINELLO, CHOISY LE ROI (FR)

(21) Appl. No.: 17/414,345

(22) PCT Filed: Dec. 19, 2019
(86) PCT No.: PCT/EP2019/086431

§ 371 Date: Jun. 16, 2021

(30) Foreign Application Priority Data
Dec. 21, 2018 (EP) 18306836.0

(51) Int. Cl. C12N 15/861; A61K 48/00

(52) U.S. Cl. CPC C12N 15/861; A61K 48/0066; C12N 2830/42; C12N 2750/14151; C12N 2750/14143

(57) ABSTRACT

The present invention relates to a recombinant expression cassette comprising a polynucleotide encoding a SMN protein. This cassette can be included in a gene therapy vector and used in a method for the treatment of spinal muscular atrophy (SMA).

### FIELD OF THE INVENTION

The present invention relates to a recombinant expression cassette comprising a SMN gene. This cassette can be included in a gene therapy vector and used in a method for the treatment of spinal muscular atrophy (SMA).

### BACKGROUND OF THE INVENTION

Spinal Muscular Atrophy (“SMA”), in its broadest sense, describes a collection of inherited and acquired central nervous system (CNS) diseases characterized by motor neuron loss in the spinal cord causing muscle weakness and atrophy. The most common form of SMA is caused by mutation of the Survival Motor Neuron (“SMN”) gene, and manifests over a wide range of severity affecting infants through adults. Infantile SMA is one of the most severe forms of this neurodegenerative disorder. The onset is usually sudden and dramatic. Some of the symptoms include: muscle weakness, poor muscle tone, weak cry, limpness or a tendency to flop, difficulty sucking or swallowing, accumulation of secretions in the lungs or throat, feeding difficulties and increased susceptibility to respiratory tract infections. The legs tend to be weaker than the arms and developmental milestones, such as lifting the head or sitting up, cannot be reached. In general, the earlier the symptoms appear, the shorter the lifespan. Shortly after symptoms appear, the motor neuron cells quickly deteriorate. The disease can be fatal. The course of SMA is directly related to the severity of weakness. Infants with a severe form of SMA frequently succumb to respiratory disease due to weakness in the muscles that support breathing. Children with milder forms of SMA live much longer, although they may need extensive medical support, especially those at the more severe end of the spectrum. Disease progression and life expectancy strongly correlate with the subject's age at onset and the level of weakness. The clinical spectrum of SMA disorders has been divided into the following five groups:
(a) Neonatal SMA (Type 0 SMA; before birth): Type 0, also known as very severe SMA, is the most severe form of SMA and begins before birth. Usually, the first symptom of type 0 is reduced movement of the fetus that is first seen between 30 and 36 weeks of the pregnancy. After birth, these newborns have little movement and have difficulties with swallowing and breathing.
(b) Infantile SMA (Type 1 SMA or Werdnig-Hoffmann disease; generally 0-6 months): Type 1 SMA, also known as severe infantile SMA or Werdnig Hoffmann disease, is very severe, and manifests at birth or within 6 months of life. Patients never achieve the ability to sit, and death usually occurs within the first 2 years without ventilatory support.
(c) Intermediate SMA (Type 2 SMA or Dubowitz disease; generally 6-18 months): Patients with Type 2 SMA, or intermediate SMA, achieve the ability to sit unsupported, but never stand or walk unaided. The onset of weakness is usually recognized sometime between 6 and 18 months. Prognosis in this group is largely dependent on the degree of respiratory involvement.
(d) Juvenile SMA (Type 3 or Kugelberg-Welander disease; generally >18 months): Type 3 SMA describes those who are able to walk independently at some point during their disease course, but often become wheelchair bound during youth or adulthood.
(e) Adult SMA (Type 4 SMA): Weakness usually begins in late adolescence in tongue, hands, or feet then progresses to other areas of the body. The course of adult disease is much slower and has little or no impact on life expectancy.

The SMA disease gene has been mapped by linkage analysis to a complex region of chromosome 5q. In humans, this region has a large inverted duplication; consequently, there are two copies of the SMN gene. SMA is caused by a recessive mutation or deletion of the telomeric copy of the gene SMN1 in both chromosomes, resulting in the loss of SMN1 gene function. However, most patients retain a centromeric copy of the gene SMN2, and its copy number in SMA patients has been implicated as having an important modifying effect on disease severity; i.e., an increased copy number of SMN2 is observed in less severe disease. Nevertheless, SMN2 is unable to compensate completely for the loss of SMN1 function, because the SMN2 gene produces reduced amounts of full-length RNA and is less efficient at making protein, although, it does so in low amounts. More particularly, the SMN1 and SMN2 genes differ by five nucleotides; one of these differences—a translationally silent C to T substitution in an exonic splicing region—results in frequent exon 7 skipping during transcription of SMN2. As a result, the majority of transcripts produced from SMN2 lack exon 7 (SMNΔEx7), and encode a truncated protein which is rapidly degraded (about 10% of the SMN2 transcripts are full length and encode a functional SMN protein).

As a consequence, gene replacement of SMN1 was proposed as a strategy for the treatment of SMA. In particular, focus was previously made on the treatment of SMA by delivery of the SMN gene across the blood-brain barrier with an AAV vector comprising an AAV9 capsid (herein after referred to as “AAV9 vector”, independently of the serotype the genome of the vector derives from) administered via the systemic route (such as in WO2010/071832). Indeed, AAV vectors comprising an AAV9 capsid were shown to be capable of crossing the blood-brain barrier and to then transduce cells involved in SMA development such as motor neurons and glial cells.

Furthermore, PCT/EP2018/068434 discloses recombinant AAV vectors comprising an AAV9 or AAVrh10 capsid, and a single-stranded genome including a gene coding spinal motor neuron (SMN) protein. This patent application also describes a number of specific constructs including a SMN gene and their unexpectedly good efficiency in treating SMA in an animal model of the disease.

It is herein disclosed further optimized constructs for the expression of SMN. These constructs provide a significant improvement of the survival rate of animals treated therewith.

### SUMMARY OF THE INVENTION

In a first aspect, the invention relates to a nucleic acid construct comprising:
a PGK promoter; and
a modified intron 2/exon 3 sequence from the human β globin gene;
a polynucleotide sequence encoding a survival of motor neuron (SMN) protein; and
a polyadenylation signal

In a particular embodiment, the PGK promoter has the sequence shown in SEQ ID NO:1, or said promoter is a functional variant of said promoter having a nucleotide sequence that is at least 80% identical to SEQ ID NO:1, in particular at least 85%, at least 90%, at least 95% or at least 99% identical to SEQ ID NO:1.

In a particular embodiment, the modified intron 2/exon 3 sequence from the human β globin gene has the sequence shown in SEQ ID NO: 12, or is a functional variant of the sequence shown in SEQ ID NO:12, which has at least 80% identity with SEQ ID NO:12, in particular at least 85%, at least 90%, at least 95% or at least 99% identity with SEQ ID NO:12.

It is herein shown that such an expression cassette compared to other expression cassettes, used in a viral vector for the correction of spinal muscular atrophy in a mouse model of this disease, led to an increase of the survival of treated animals at level that was never reported before.

In a particular embodiment of the first aspect, the polyadenylation signal is selected in the group consisting of the SMN1 gene polyadenylation signal, a polyadenylation signal from the human β globin gene (HBB pA), the bovine growth hormone polyadenylation signal, the SV40 polyadenylation signal, and a synthetic polyA, such as the synthetic polyA of SEQ ID NO:10. In a particular embodiment of the first aspect, the polyadenylation signal is a HBB polyadenylation signal, such as a HBB polyadenylation signal having a sequence selected in the group consisting of SEQ ID NO: 7 and SEQ ID NO: 8, or a functional variant thereof having a nucleotide sequence that is at least 80% identical to the sequence shown in SEQ ID NO: 7 or SEQ ID NO: 8, in particular at least 85%, at least 90%, at least 95% or at least 99% identical to SEQ ID NO:7 or SEQ ID NO:8.

In a particular embodiment, the polynucleotide sequence (ORF) encoding a SMN protein is derived from the human SMN1 gene.

In a particular embodiment, the expression cassette can be flanked by sequences suitable for the packaging of the expression cassette into a recombinant viral vector. For example, the expression cassette can be flanked by an AAV 5′-ITR and an AAV 3′-ITR for its further packaging into an AAV vector or by a 5′-LTR and a 3′-LTR for its further packaging into a retroviral vector, such as into a lentiviral vector.

In a particular embodiment, the expression cassette has a sequence comprising or consisting of the sequence shown in SEQ ID NO:11, or a sequence that is at least 80% identical to SEQ ID NO:11, e.g. at least 85% identical, at least 86% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical or at least 99% identical to SEQ ID NO:11.

In a second aspect, the invention relates to a recombinant vector comprising the expression cassette of the invention.

In a particular embodiment, the vector is a plasmid vector. A plasmid vector may comprise the expression cassette flanked or not flanked by sequences suitable for the packaging of the expression cassette into a recombinant viral vector.

In another particular embodiment, the vector is a recombinant viral vector. Illustrative viral vectors useful in the practice of the invention comprise, without limitation, adeno-associated (AAV) vectors, lentiviral vectors and adenoviral vectors. In another particular embodiment, the recombinant vector of the invention is a recombinant AAV (rAAV) vector. In a further embodiment, the rAAV vector has a capsid selected in the group consisting of an

AAV1, AAV2, AAV3, AAV4, AAVS, AAV6, AAV7, AAV8, AAV9, AAVrh10, AAV11, AAV12 and AAV-PHP.B capsid. In another particular embodiment, the rAAV vector has a capsid selected from an AAV9 and an AAVrh10 capsid. The rAAV vector of the invention can have a single-stranded or double-stranded, self-complementary genome. The genome of the rAAV vector can be derived from any AAV genome, meaning that its AAV 5′-ITR and AAV 3′-ITR can be derived from any AAV serotype, the AAV 5′- and 3′-ITRs being more particularly derived from AAV1, AAV2, AAV3, AAV4, AAV5, AAV6, AAV7, AAV8, AAV9, AAVrh10, AAV11, AAV12, or AAV-PHP.B capsid 5′- and 3′-ITRs. In a particular embodiment, the AAV 5′- and 3′-ITRs are AAV2 5′- and 3′-ITRs. In the practice of the present invention, the AAV capsid and the AAV ITRs may be derived from the same serotype or different serotypes. When the serotypes of the capsid and the genome are different, the rAAV vector is referred to as “pseudotyped”. In a particular embodiment, the rAAV vector of the invention is a pseudotyped vector.

In yet another aspect, the invention relates to the vector of the invention, for use in a method for the treatment of a disease by gene therapy. In a particular embodiment, the transgene of interest is a gene coding a SMN protein and the disease is spinal muscular atrophy (SMA), such as infantile SMA, intermediate SMA, juvenile SMA or adult-onset SMA. In a particular embodiment, the vector for use according to the invention is a rAAV vector as disclosed herein. In another embodiment, said rAAV vector is for administration into the cerebrospinal fluid of a subject, in particular by intrathecal and/or intracerebroventricular injection. Alternatively, said rAAV vector is for peripheral administration, such as for intravascular (e.g. intravenous or intra-arterial), intramuscular and intraperitoneal administration.

### BRIEF DESCRIPTION OF THE DRAWINGS

FIG. 1: Kaplan-Meyer survival curve of untreated Smn^{2B/-} mice, wild-type animals (n=10 mice per group) and Smn^{2B/-} mice treated with different single-stranded AAV vectors comprising the hSMN1 transgene.

FIG. 2: body weight assessment of untreated Smn^{2B/-} mice, wild-type animals (n=10 mice per group) and Smn^{2B/-} mice treated with a single-stranded AAV vector comprising the hSMN1 transgene operably linked to the PGK promoter and a modified intron 2/exon 3 sequence from the human β globin gene.

FIG. 3: Kaplan-Meyer survival curve of untreated Smn^{2B/-} mice, wild-type animals (n=10 mice per group) and Smn^{2B/-} mice treated with different doses of the ssAAV9-7212 vector.

FIG. 4: body weight assessment of untreated Smn^{2B/-} mice, wild-type animals (n=10 mice per group) and Smn^{2B/-} mice treated with different doses of the ssAAV9-7212 vector.

### DETAILED DESCRIPTION OF THE INVENTION

The present invention provides materials and methods useful in therapy, more particularly for the treatment of SMA. More specifically, the present invention provides combinations of regulatory elements useful for the improved expression of transgenes of interest, such as a gene encoding a SMN protein. The advantages of the invention are more particularly shown with respect to the treatment of SMA. Indeed, the inventors have shown an impressive improvement of the survival of an animal model SMA, the level of which was never reported before.

### Expression Cassette

The invention relates, in a first aspect, to an expression cassette comprising, in this order from 5′ to 3′:
a PGK promoter;
a modified intron 2/exon 3 sequence from the human β globin gene;
a polynucleotide sequence of interest encoding a SMN protein; and
a polyadenylation signal.

The PGK promoter has been described in Singer et al., Gene, 32 (1984), p. 409). Its sequence is shown in SEQ ID NO: 1. Unexpectedly, it is herein shown that the PGK promoter combined to a modified intron 2/exon 3 sequence from the human β-globin gene, when operatively linked to a transgene of interest such as a SMN transgene, and compared to other ubiquitous promoters for the expression of a SMN protein, provides largely better survival rate in a mouse model of SMA.

In a particular embodiment, the PGK promoter is a variant of the sequence shown in SEQ ID NO:1, having a nucleotide sequence that is at least 80% identical to the sequence shown in SEQ ID NO:1, in particular at least 85%, at least 90%, at least 95% or at least 99% identical to SEQ ID NO:1. In the context of the present invention, a functional variant of the PGK promoter is a sequence deriving therefrom by one or more nucleotide modifications, such as nucleotide substitution, addition or deletion, that results in the same or substantially the same level of expression (e.g. ±20%, such as ±10%, ±5% or ±1%) of the SMN transgene operatively linked thereto.

The expression cassette comprises a sequence composed of a modified intron 2/exon 3 sequence from the human β globin gene. This sequence is located 3′ of the PGK promoter and 5′ of the transgene coding SMN protein.

In a particular embodiment, the modified intron 2/exon 3 sequence from the human p globin gene has the sequence shown in SEQ ID NO: 12, or is a functional variant of the sequence shown in SEQ ID NO:12, which has at least 80% identity with SEQ ID NO:12, in particular at least 85%, at least 90%, at least 95% or at least 99% identity with SEQ ID NO:12. In the context of the present invention, a functional variant of the modified intron 2/exon 3 sequence from the human β globin gene is a sequence deriving therefrom by one or more nucleotide modifications, such as nucleotide substitution, addition or deletion, that results in the same or substantially the same level of expression (e.g. ±20%, such as ±10%, ±5% or ±1%) of the SMN transgene operatively linked thereto.

The polyadenylation signal in the expression cassette of the invention may be derived from a number of genes. Illustrative polyadenylation signals include, without limitation, the

SMN1 gene polyadenylation signal, the human β globin gene (HBB) polyadenylation signal, the bovine growth hormone polyadenylation signal and the SV40 polyadenylation signal. In a particular embodiment, the polyadenylation signal is a HBB polyadenylation signal, such as a HBB polyadenylation signal having a sequence selected in the group consisting of SEQ ID NO: 7 and SEQ ID NO: 8.

In a particular embodiment, the HBB polyadenylation signal is a functional variant of the sequence shown in SEQ ID NO:7 or SEQ ID NO:8, which has at least 80% identity with SEQ ID NO:7 or SEQ ID NO:8, in particular at least 85%, at least 90%, at least 95% or at least 99% identity with SEQ ID NO:7 or SEQ ID NO:8. In the context of the present invention, a functional variant of the HBB polyadenylation signal is a sequence deriving therefrom by one or more nucleotide modifications, such as nucleotide substitution, addition or deletion, that results in the same or substantially the same level of expression (e.g. ±20%, such as ±10%, ±5% or ±1%) of the SMN transgene operatively linked thereto.

Of course, other sequences such as a Kozak sequence (such as that shown in SEQ ID NO:9) are known to those skilled in the art and are introduced to allow expression of a transgene.

The expression cassette disclosed herein can be flanked by sequences suitable for the packaging of the expression cassette into a recombinant viral vector. For example, the expression cassette can be flanked by an AAV 5′-ITR and an AAV 3′-ITR for its further packaging into an AAV vector or by a 5′-LTR and a 3′-LTR for its further packaging into a retroviral vector, such as into a lentiviral vector.

In a preferred embodiment, the transgene of interest encoding a SMN protein is a human SMN protein. In a particular embodiment, the nucleic acid coding the human SMN protein is derived from the sequence having the Genbank accession No. NM_000344.3. In a particular embodiment, the gene encoding the SMN protein consists of or comprises the sequence shown in SEQ ID NO: 2.

In another particular embodiment, the sequence of the transgene encoding the SMN protein, in particular the human SMN protein, is optimized. Sequence optimization may include a number of changes in a nucleic acid sequence, including codon optimization, increase of GC content, decrease of the number of CpG islands, decrease of the number of alternative open reading frames (ARFs) and/or decrease of the number of splice donor and splice acceptor sites. Because of the degeneracy of the genetic code, different nucleic acid molecules may encode the same protein. It is also well known that the genetic codes of different organisms are often biased towards using one of the several codons that encode the same amino acid over the others. Through codon optimization, changes are introduced in a nucleotide sequence that take advantage of the codon bias existing in a given cellular context so that the resulting codon optimized nucleotide sequence is more likely to be expressed in such given cellular context at a relatively high level compared to the non-codon optimised sequence. In a preferred embodiment of the invention, such sequence optimized nucleotide sequence encoding a SMN protein, is codon-optimized to improve its expression in human cells compared to non-codon optimized nucleotide sequences coding for the same protein (e.g. a SMN protein), for example by taking advantage of the human specific codon usage bias.

In a particular embodiment, the optimized coding sequence (e.g. a SMN coding sequence) is codon optimized, and/or has an increased GC content and/or has a decreased number of alternative open reading frames, and/or has a decreased number of splice donor and/or splice acceptor sites, as compared to the wild-type coding sequence (such as the wild-type human SMN1 coding sequence of SEQ ID NO: 2).

In a particular embodiment, the nucleic acid sequence encoding the SMN protein is at least 70% identical, in particular at least 75% identical, at least 80% identical, at least 85% identical, at least 86% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical or at least 99% identical to the sequence shown in SEQ ID NO: 2.

As mentioned above, in addition to the GC content and/or number of ARFs, sequence optimization may also comprise a decrease in the number of CpG islands in the sequence and/or a decrease in the number of splice donor and acceptor sites. Of course, as is well known to those skilled in the art, sequence optimization is a balance between all these parameters, meaning that a sequence may be considered optimized if at least one of the above parameters is improved while one or more of the other parameters is not, as long as the optimized sequence leads to an improvement of the transgene, such as an improved expression and/or a decreased immune response to the transgene in vivo.

In addition, the adaptiveness of a nucleotide sequence encoding a SMN protein to the codon usage of human cells may be expressed as codon adaptation index (CAI). A codon adaptation index is herein defined as a measurement of the relative adaptiveness of the codon usage of a gene towards the codon usage of highly expressed human genes. The relative adaptiveness (w) of each codon is the ratio of the usage of each codon, to that of the most abundant codon for the same amino acid. The CAI is defined as the geometric mean of these relative adaptiveness values. Non-synonymous codons and termination codons (dependent on genetic code) are excluded. CAI values range from 0 to 1, with higher values indicating a higher proportion of the most abundant codons (see Sharp and Li, 1987, Nucleic Acids Research 15: 1281-1295; also see: Kim et al, Gene. 1997, 199:293-301; zur Megede et al, Journal of Virology, 2000, 74: 2628-2635).

In a particular embodiment, the transgene of interest encodes a human SMN protein, and the nucleic acid sequence coding for human SMN protein consists of or comprises an optimized sequence as sequence shown in SEQ ID NO: 3, SEQ ID NO: 4, SEQ ID NO: 5 or SEQ ID NO: 6.

The expression cassette disclosed herein can be flanked by sequences suitable for the packaging of the expression cassette into a recombinant viral vector. For example, the expression cassette can be flanked by an AAV 5′-ITR and an AAV 3′-ITR for its further packaging into an AAV vector or by a 5′-LTR and a 3′-LTR for its further packaging into a retroviral vector, such as into a lentiviral vector.

### Recombinant Vectors

The expression cassette of the invention can be included in a recombinant vector. The invention thus further relates to a recombinant vector comprising an expression cassette as described above.

In a particular embodiment, the recombinant vector is a plasmid vector. In particular, a plasmid vector may comprise the expression cassette flanked or not flanked by sequences suitable for the packaging of the expression cassette into a recombinant viral vector as described above.

In another particular embodiment, the vector is a recombinant viral vector. Illustrative viral vectors useful in the practice of the invention comprise, without limitation, adeno-associated (AAV) vectors, lentiviral vectors and adenoviral vectors.

In another particular embodiment, the recombinant vector of the invention is a recombinant AAV (rAAV) vector.

The human parvovirus Adeno-Associated Virus (AAV) is a dependovirus that is naturally defective for replication, which is able to integrate into the genome of the infected cell to establish a latent infection. AAV vectors have arisen considerable interest as potential vectors for human gene therapy. Among the favorable properties of the virus are its lack of association with any human disease, its ability to infect both dividing and non-dividing cells, and the wide range of cell lines derived from different tissues that can be infected.

In the context of the present invention, the terms “adeno-associated virus” (AAV) and “recombinant adeno-associated virus” (rAAV) are used interchangeably herein and refer to an AAV whose genome was modified, as compared to a wild-type (wt) AAV genome, by replacement of a part of the wt genome with a transgene of interest. The term “transgene” refers to a gene whose nucleic acid sequence is non-naturally occurring in an AAV genome. In particular, the rAAV vector is to be used in gene therapy. As used herein, the term “gene therapy” refers to the transfer of genetic material (e.g., DNA or RNA) of interest into a host to treat or prevent a genetic or acquired disease or condition. The genetic material of interest encodes a product (e.g., a polypeptide or functional RNA) whose production is desired in vivo. For example, the genetic material of interest can encode a hormone, receptor, enzyme or polypeptide of therapeutic value. Alternatively, the genetic material of interest can encode a functional RNA of therapeutic value, such as an antisense RNA or a shRNA of therapeutic value.

Recombinant AAVs may be engineered using conventional molecular biology techniques, making it possible to optimize these particles for cell specific delivery of nucleic acid sequences, for minimizing immunogenicity, for tuning stability and particle lifetime, for efficient degradation, for accurate delivery to the nucleus. Desirable AAV elements for assembly into vectors include the cap proteins, including the vp1, vp2, vp3 and hypervariable regions, the rep proteins, including rep 78, rep 68, rep 52, and rep 40, and the sequences encoding these proteins. These elements may be readily used in a variety of vector systems and host cells.

In the present invention, the capsid of the AAV vector may be derived from a naturally or non-naturally-occurring serotype. In a particular embodiment, the serotype of the capsid of the AAV vector is selected from AAV natural serotypes. Alternatively to using AAV natural serotypes, artificial AAV serotypes may be used in the context of the present invention, including, without limitation, AAV with a non-naturally occurring capsid protein. Such an artificial capsid may be generated by any suitable technique, using a selected AAV sequence (e.g., a fragment of a vp1 capsid protein) in combination with heterologous sequences which may be obtained from a different selected AAV serotype, non-contiguous portions of the same AAV serotype, from a non-AAV viral source, or from a non-viral source. A capsid from an artificial AAV serotype may be, without limitation, a chimeric AAV capsid, a recombinant AAV capsid, or a “humanized” AAV capsid.

According to a particular embodiment, the capsid of the AAV vector is of the AAV-1, -2, AAV-2 variants (such as the quadruple-mutant capsid optimized AAV-2 comprising an engineered capsid with Y44+500+730F+T491V changes, disclosed in Ling et al., 2016 Jul. 18, Hum Gene Ther Methods. [Epub ahead of print]), -3 and AAV-3 variants (such as the AAV3-ST variant comprising an engineered AAV3 capsid with two amino acid changes, S663V+T492V, disclosed in Vercauteren et al., 2016, Mol. Ther. Vol. 24(6), p. 1042), -3B and AAV-3B variants, -4, -5, -6 and AAV-6 variants (such as the AAV6 variant comprising the triply mutated AAV6 capsid Y731F/Y705F/T492V form disclosed in Rosario et al., 2016, Mol Ther Methods Clin Dev. 3, p.16026), -7, -8, -9 and AAV-9 variants (such as AAVhu68), -2G9, -10 such as -cy10 and -rh10, -11, -12, -rh39, -rh43, -rh74, -dj, Anc80L65, LK03, AAV.PHP.B, AAV2i8, porcine AAV such as AAVpo4 and AAVpo6, and tyrosine, lysine and serine capsid mutants of AAV serotypes. In addition, the capsid of other non-natural engineered variants (such as AAV-spark100), chimeric AAV or AAV serotypes obtained by shuffling, rationale design, error prone PCR, and machine learning technologies can also be useful.

In a particular embodiment, the AAV vector has a naturally occurring capsid, such as an AAV1, AAV2, AAV3, AAV4, AAVS, AAV6, AAV7, AAV8, AAV9, AAV-cy10, AAVrh10, AAV11 and AAV12 capsid. In a particular embodiment, the capsid of the AAV vector is selected from an AAV9 or AAVrh10 capsid.

In a particular embodiment, the AAV vector is an AAV vector with high tropism to motoneurons, glial cells, muscle cells and/or cardiac cells. In a variant of this embodiment, the AAV vector has an AAV8, AAV9, AAVrh10, PHP.B or AAV Anc80L65 capsid.

In particular embodiments of the invention, a rAAV vector may comprise an AAV9 or AAVrh10 capsid. Such vector is herein termed “AAV9 vector” or “AAVrh10 vector”, respectively, independently of the serotype the genome contained in the rAAV vector is derived from. Accordingly, an AAV9 vector may be a vector comprising an AAV9 capsid and an AAV9 derived genome (i.e. comprising AAV9 ITRs) or a pseudotyped vector comprising an AAV9 capsid and a genome derived from a serotype different from the AAV9 serotype. Likewise, an AAVrh10 vector may be a vector comprising an AAVrh10 capsid and an AAVrh10 derived genome (i.e. comprising AAVrh10 ITRs) or a pseudotyped vector comprising an AAVrh10 capsid and a genome derived from a serotype different from the AAVrh10 serotype.

The genome present within the rAAV vector of the present invention may be single-stranded or self-complementary. In the context of the present invention a “single stranded genome” is a genome that is not self-complementary, i.e. the coding region contained therein has not been designed as disclosed in McCarty et al., 2001 and 2003 (Op. cit) to form an intra-molecular double-stranded DNA template. On the contrary, a “self-complementary AAV genome” has been designed as disclosed in McCarty et al., 2001 and 2003 (Op. cit) to form an intra-molecular double-stranded DNA template.

In a particular embodiment, the rAAV genome is a single stranded genome.

The genome present within the rAAV vector may preferably AAV rep and cap genes, and comprises a transgene of interest. Therefore, the AAV genome may comprise a transgene of interest flanked by AAV ITRs. The ITRs may be derived from any AAV genome, such as an AAV1, AAV2, AAV3, AAV4, AAV5, AAV6, AAV7, AAV8, AAV9, AAV-cy10, AAVrh10, AAV11 or AAV12 genome. In a particular embodiment, the genome of the AAV vector comprises 5′- and 3′-AAV2 ITRs.

Any combination of AAV serotype capsid and ITR may be implemented in the context of the present invention, meaning that the AAV vector may comprise a capsid and ITRs derived from the same AAV serotype, or a capsid derived from a first serotype and ITRs derived from a different serotype than the first serotype. Such a vector with capsid ITRs deriving from different serotypes is also termed a “pseudotyped vector”. More particularly, the pseudotyped rAAV vector can include:
a genome comprising AAV1 5′- and 3′-ITRs, and a capsid selected in the group consisting of an AAV2, AAV3, AAV4, AAV5, AAV6, AAV7, AAV8, AAV9, AAVrh10, AAV11 and AAV12 capsid;
a genome comprising AAV2 5′- and 3′-ITRs, and a capsid selected in the group consisting of an AAV1, AAV3, AAV4, AAV5, AAV6, AAV7, AAV8, AAV9, AAVrh10, AAV11 and AAV12 capsid;
a genome comprising AAV3 5′- and 3′-ITRs, and a capsid selected in the group consisting of an AAV1, AAV2, AAV4, AAV5, AAV6, AAV7, AAV8, AAV9, AAVrh10, AAV11 and AAV12 capsid;
a genome comprising AAV4 5′- and 3′-ITRs, and a capsid selected in the group consisting of an AAV1, AAV2, AAV3, AAV5, AAV6, AAV7, AAV8, AAV9, AAVrh10, AAV11 and AAV12 capsid;
a genome comprising AAV5 5′- and 3′-ITRs, and a capsid selected in the group consisting of an AAV1, AAV2, AAV3, AAV4, AAV6, AAV7, AAV8, AAV9, AAVrh10, AAV11 and AAV12 capsid;
a genome comprising AAV6 5′- and 3′-ITRs, and a capsid selected in the group consisting of an AAV1, AAV2, AAV3, AAV4, AAV5, AAV7, AAV8, AAV9, AAVrh10, AAV11 and AAV12 capsid;
a genome comprising AAV7 5′- and 3′-ITRs, and a capsid selected in the group consisting of an AAV1, AAV2, AAV3, AAV4, AAV5, AAV6, AAV8, AAV9, AAVrh10, AAV11 and AAV12 capsid;
a genome comprising AAV8 5′- and 3′-ITRs, and a capsid selected in the group consisting of an AAV1, AAV2, AAV3, AAV4, AAV5, AAV6, AAV7, AAV9, AAVrh10, AAV11 and AAV12 capsid;
a genome comprising AAV9 5′- and 3′-ITRs, and a capsid selected in the group consisting of an AAV1, AAV2, AAV3, AAV4, AAV5, AAV6, AAV7, AAV8, AAVrh10, AAV11 and AAV12 capsid;
a genome comprising AAVrh10 5′- and 3′-ITRs, and a capsid selected in the group consisting of an AAV1, AAV2, AAV3, AAV4, AAV5, AAV6, AAV7, AAV8, AAV9, AAV11 and AAV12 capsid; or
a genome comprising AAV11 5′- and 3′-ITRs, and a capsid selected in the group consisting of an AAV1, AAV2, AAV3, AAV4, AAV5, AAV6, AAV7, AAV8, AAV9, AAVrh10, and AAV12 capsid.
   In a particular embodiment, the pseudotyped rAAV vector includes a genome, in particular a single-stranded genome, comprising AAV2 5′- and 3′-ITRs, and a capsid selected in the group consisting of an AAV1, AAV3, AAV4, AAV5, AAV6, AAV7, AAV8, AAV9, AAVrh10, AAV11 and AAV12 capsid. In another particular embodiment, the pseudotyped rAAV vector includes a genome, in particular a single-stranded genome, comprising AAV2 5′- and 3′-ITRs, and a capsid selected in the group consisting of an AAV9 and AAVrh10 capsid.

In a particular embodiment, in particular in a variant wherein the genome is a single-stranded AAV genome (which is not self-complementary as explained above), the expression cassette has a size comprised between 2100 and 4400 nucleotides, in particular between 2700 and 4300 nucleotides, more particularly between 3200 and 4200 nucleotides. In a particular embodiment, the size of the expression cassette is of about 3200 nucleotides, about 3300 nucleotides, about 3400 nucleotides, about 3500 nucleotides, about 3600 nucleotides, about 3700 nucleotides, about 3800 nucleotides, about 3900 nucleotides, about 4000 nucleotides, about 4100 nucleotides, or about 4200 nucleotides.

According to the present invention, the term “about”, when referring to a numerical value, means plus or minus 5% of this numerical value.

In another aspect, the invention provides DNA plasmids comprising rAAV genomes of the invention. Production of rAAV requires that the following components are present within a single cell (denoted herein as a packaging cell): a rAAV genome, AAV rep and cap genes separate from (i.e., not in) the rAAV genome, and helper virus functions. Production of pseudotyped rAAV is disclosed in, for example, WO 01/83692. Production may implement transfection a cell with two, three or more plasmids. For example three plasmids may be used, including: (i) a plasmid carrying a Rep/Cap cassette, (ii) a plasmid carrying the rAAV genome (i.e. a transgene flanked with AAV ITRs) and (iii) a plasmid carrying helper virus functions (such as adenovirus helper functions). In another embodiment, a two-plasmid system may be used, comprising (i) a plasmid comprising Rep and Cap genes, and helper virus functions, and (ii) a plasmid comprising the rAAV genome.

In a further aspect, the invention relates to a plasmid comprising the isolated nucleic acid construct of the invention. This plasmid may be introduced in a cell for producing a rAAV vector according to the invention by providing the rAAV genome to said cell.

A method of generating a packaging cell is to create a cell line that stably expresses all the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are incorporated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

General principles of rAAV production are reviewed in, for example, Carter, 1992, Current Opinions in Biotechnology, 1533-539; and Muzyczka, 1992, Curr. Topics in Microbial, and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); McLaughlin et al., J. Virol., 62: 1963 (1988); and Lebkowski et al., 1988 Mol. Cell. Biol., 7:349 (1988); Samulski et al. (1989, J. Virol., 63:3822-3828); U.S. Pat. No. 5,173,414; WO 95/13365 and corresponding U.S. Pat. No. 5,658.776 ; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13: 1244- 1250; Paul et al. (1993) Human Gene Therapy 4:609-615; Clark et al. (1996) Gene Therapy 3: 1124-1132; U.S. Pat. No. 5,786,211; U.S. Pat. No. 5,871,982; and U.S. Pat. No. 6,258,595. The invention thus also provides packaging cells that produce infectious rAAV. In one embodiment packaging cells may be stably transformed cancer cells such as HeLa cells, HEK293 cells, HEK 293T, HEK293vc and PerC.6 cells (a cognate 293 line). In another embodiment, packaging cells are cells that are not transformed cancer cells such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

The rAAV may be purified by methods standard in the art such as by column chromatography or cesium chloride gradients. Methods for purifying rAAV vectors from helper virus are known in the art and include methods disclosed in, for example, Clark et ah, Hum. Gene Ther., 10(6): 1031-1039 (1999); Schenpp and Clark, Methods Mol. Med., 69: 427-443 (2002); U.S. Pat. No. 6,566,118 and WO 98/09657.

In another aspect, the invention provides compositions comprising a rAAV disclosed in the present application. Compositions of the invention comprise rAAV in a pharmaceutically acceptable carrier. The compositions may also comprise other ingredients such as diluents and adjuvants. Acceptable carriers, diluents and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics or polyethylene glycol (PEG).

### Therapeutic Uses of the Invention

Thanks to the present invention, the transgene encoding SMN protein interest may be expressed efficiently in a tissue of interest for the treatment of spinal muscular atrophy (SMA), such as SMA is infantile SMA, intermediate SMA, juvenile SMA or adult-onset SMA

Accordingly, the invention relates to a vector as disclosed herein, for use in therapy.

In a particular embodiment wherein the transgene of interest encodes a SMN protein, said transgene may be delivered to lower motor neurons, such as to spinal cord motor neurons (i.e. motor neurons whose soma is within the spinal cord) and to spinal cord glial cells. in this embodiment, the vector of the invention may be used in a method for the treatment of SMA. In a particular embodiment, SMA is neonatal SMA, infantile SMA, intermediate SMA, juvenile SMA or adult-onset SMA

In a preferred embodiment, the vector of the invention may be an AAV9 or AAVrh10 vector comprising a genome as defined above, such as a single-stranded genome, comprising as a transgene of interest a gene coding a SMN protein.

The vector for use according to the invention may be administered locally with or without systemic co-delivery. In the context of the present invention, local administration denotes an administration into the cerebrospinal fluid of the subject, such as via an intrathecal injection of the rAAV vector. In some embodiment, the methods further comprise administrating an effective amount of the vector by intracerebral administration. In some embodiment, the vector may be administrated by intrathecal administration and by intracerebral administration. In some embodiment the vector may be administrated by a combined intrathecal and/or intracerebral and/or peripheral (such as a vascular, for example intravenous or intra-arterial, in particular intravenous) administration.

As used herein the term “intrathecal administration” refers to the administration of a vector according to the invention, or a composition comprising a vector of the invention, into the spinal canal. For example, intrathecal administration may comprise injection in the cervical region of the spinal canal, in the thoracic region of the spinal canal, or in the lumbar region of the spinal canal. Typically, intrathecal administration is performed by injecting an agent, e.g., a composition comprising a vector of the invention, into the subarachnoid cavity (subarachnoid space) of the spinal canal, which is the region between the arachnoid membrane and pia mater of the spinal canal. The subarachnoid space is occupied by spongy tissue consisting of trabeculae (delicate connective tissue filaments that extend from the arachnoid mater and blend into the pia mater) and intercommunicating channels in which the cerebrospinal fluid is contained. In some embodiments, intrathecal administration is not administration into the spinal vasculature. In certain embodiment the intrathecal administration is in the lumbar region of the subject

As used herein, the term “intracerebral administration” refers to administration of an agent into and/or around the brain. Intracerebral administration includes, but is not limited to, administration of an agent into the cerebrum, medulla, pons, cerebellum, intracranial cavity, and meninges surrounding the brain. Intracerebral administration may include administration into the dura mater, arachnoid mater, and pia mater of the brain. Intracerebral administration may include, in some embodiments, administration of an agent into the cerebrospinal fluid (CSF) of the subarachnoid space surrounding the brain. Intracerebral administration may include, in some embodiments, administration of an agent into ventricles of the brain/forebrain, e.g., the right lateral ventricle, the left lateral ventricle, the third ventricle, the fourth ventricle. In some embodiments, intracerebral administration is not administration into the brain vasculature.

In some embodiments, intracerebral administration involves injection using stereotaxic procedures. Stereotaxic procedures are well known in the art and typically involve the use of a computer and a 3-dimensional scanning device that are used together to guide injection to a particular intracerebral region, e.g., a ventricular region. Micro-injection pumps (e.g., from World Precision Instruments) may also be used. In some embodiments, a microinjection pump is used to deliver a composition comprising a vector of the invention. In some embodiments, the infusion rate of the composition is in a range of 1 μl/minute to 100 μl/minute. As will be appreciated by the skilled artisan, infusion rates will depend on a variety of factors, including, for example, species of the subject, age of the subject, weight/size of the subject, the kind of vector (i.e. plasmid or viral vector, type of viral vector, serotype of the vector in case of a rAAV vector), dosage required, intracerebral region targeted, etc. Thus, other infusion rates may be deemed by a skilled artisan to be appropriate in certain circumstances.

Furthermore, thanks to the capacity to cross the blood-brain barrier elicited by certain rAAV vectors (e.g. rAAV9 or rAAVrh10 vector) administration via a systemic route may be considered. Accordingly, methods of administration of the rAAV vector include but are not limited to, intramuscular, intraperitoneal, vascular (e.g. intravenous or intra-arterial), subcutaneous, intranasal, epidural, and oral routes. In a particular embodiment, the systemic administration is a vascular injection of the rAAV vector, in particular an intravenous injection.

In a particular embodiment, the vector is administered into the cerebrospinal fluid, in particular by intrathecal injection. In a particular embodiment, the patient is put in the Trendelenburg position after intrathecal delivery of an rAAV vector.

The amount of the vector of the invention which will be effective in the treatment of SMA can be determined by standard clinical techniques. In addition, in vivo and/or in vitro assays may optionally be employed to help predict optimal dosage ranges. The dosage of the vector of the invention administered to the subject in need thereof will vary based on several factors including, without limitation, the specific type or stage of the disease treated, the subject's age or the level of expression necessary to obtain the therapeutic effect. One skilled in the art can readily determine, based on its knowledge in this field, the dosage range required based on these factors and others. Typical doses of AAV vectors are of at least 1×10⁸ vector genomes per kilogram body weight (vg/kg), such as at least 1×10⁹ vg/kg, at least 1×10¹⁰ vg/kg, at least 1×10¹¹ vg/kg, at least 1×10¹² vg/kg at least 1×10¹³ vg/kg, at least 1×10¹⁴ vg/kg or at least 1×10¹⁵ vg/kg.

### EXAMPLES

### Example 1

It is herein demonstrated that survival of a mouse model of SMA is greatly improved, beyond expectation, after administration of an AAV vector carrying a human SMN1 gene operably linked to a PGK promoter and a modified intron 2/exon 3 sequence from the human β globin gene as defined above as compared to AAV vectors comprising other combinations of regulatory elements.

### Materials and Methods

### Vector Production

The AAV vector according to the invention (also referred to as the 7212 vector) used is a single-stranded recombinant AAV9 vector carrying human SMN1 gene under the control of the PGK promoter, modified intron 2/exon 3 sequence from the human β globin gene and a polyA region from the HBB gene.

The ssAAV9 vector was produced by the tri-transfection system using standard procedures (Xiao et al., J. Virol. 1998; 72:2224-2232). Pseudo-typed recombinant rAAV2/9 (rAAV9) viral preparations were generated by packaging AAV2-inverted terminal repeat (ITR) recombinant genomes into AAV9 capsids. Briefly, the cis-acting plasmid carrying the PGK-hSMN1 construct, a trans-complementing rep-cap9 plasmid encoding the proteins necessary for the replication and structure of the vector and an adenovirus helper plasmid were co-transfected into HEK293 cells. Vector particles were purified through two sequential cesium chloride gradient ultra-centrifugations and dialyzed against sterile PBS-MK. DNAse I resistant viral particles were treated with proteinase K. Viral titres were quantified by a TaqMan real-time PCR assay (Applied Biosystem) with primers and probes specific for the polyA region and expressed as viral genomes per ml (vg/ml).

This vector was compared to AAV vectors having a single-stranded genome comprising the following elements:
Vector 7209: plasmid carrying the CAG promoter (a hybrid CMV enhancer/chicken-β-actin promoter and beta-globin splice acceptor site), human SMN1 gene, human SMN1 3′-UTR and a polyA region from the HBB gene;
Vector 7210: the vector of example 1, carrying the CAG promoter, human SMN1 gene, and a polyA region from the HBB gene;
Vector 7211: vector carrying the CAG promoter, human SMN1 gene, a Woodchuck hepatitis virus posttranscriptional regulatory element (WPRE) and a polyA region from the HBB gene.

### Animals

Smn^{2B/-} mice were obtained by two colonies crossing Smn^{2B/2B} homozygous (kindly provided by Rashmi Kothary, Ottawa, Ontario, Canada) and Smn^{+/-} heterozygous mice (Jackson Laboratories) were mated to generate Smn^{2B/+} and Smn^{2B/-} mice. Litters were genotyped at birth. Mice were kept under a 12-hour light 12-hour dark cycle and fed with a standard diet supplemented with Diet Recovery gel, food and water ad libitum. Care and manipulation of mice were performed in accordance with national and European legislations on animal experimentation and approved by the institutional ethical committee.

### In Vivo Gene Therapy

Smn^{2B/-} mice were treated with viral particles at birth (P0) by intracerebroventricular (ICV) injections; ssAAV9-hSMN1 (8×10e¹² vg/kg, 7 μl total volume) was administrated into the right lateral ventricle. Control Smn^{2B/+} littermates and wild-type mice received 7 μl of PBS-MK (1 mM MgCl₂, 2.5 mM KCl) at birth using the same procedure.

### Results

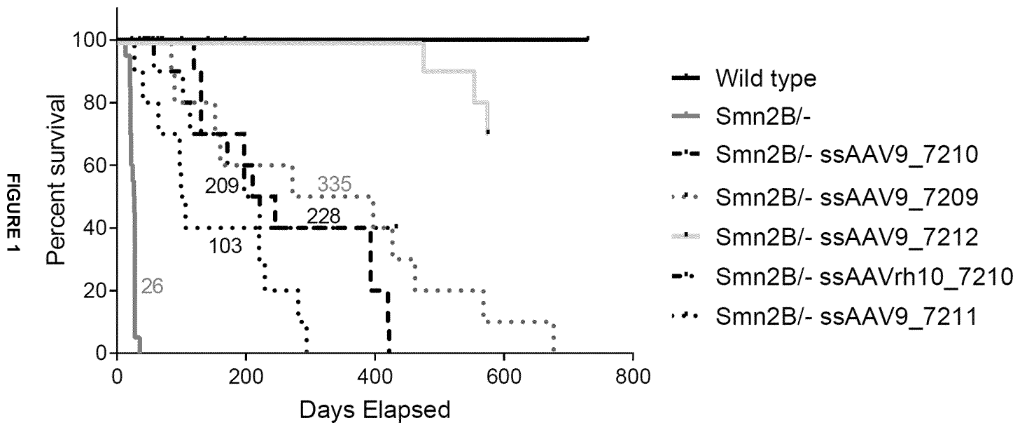

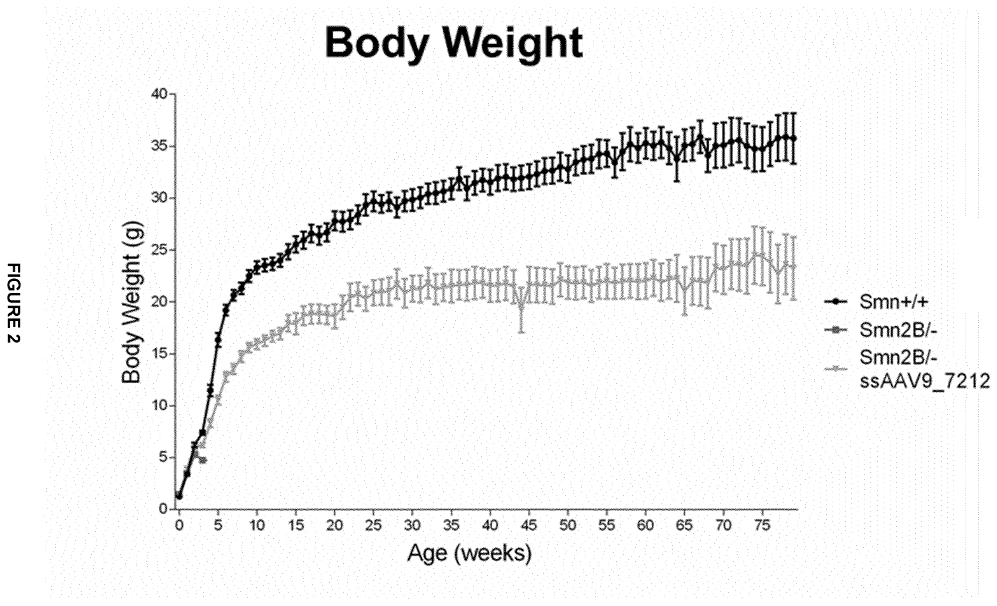

The results are presented in FIGS. 1 and 2.

The aim of the study is to assess the therapeutic efficacy of single-stranded (ss)AAV9 vectors that express human SMN1 in a mouse model of spinal muscular atrophy. We compared the effect of four ssAAV9-hSM Ni vectors by intracerebroventricular (ICV) administration in Smn^{2B/-} newborn mice 21 and 90 days post-injection.

We analyzed different parameters:
Survival,
Body weight,
spinal motor neuron counting

Four ssAAV9-hSMN1 vectors (7209, 7210, 7211 and 7212, the latter being according to the invention) and one ssAAVrh10-hSMN1 vector containing the wild-type human SMN1 coding sequence (NCBI Reference Sequence: NM_000344.3) and different promoters and regulatory sequences were produced by the tri-transfection system in HEK293 cells.

We administered the vectors into the cerebrospinal fluid of Smn^{2B/-} newborn mice (post-natal day 0-1-P0/1 by ICV injection). Smn^{2B/-} mice develop a severe phenotype with body weight loss and clinical signs of the disease at around 15 days of age; the current mean survival of Smn^{2B/-} mice of our colony is 26 days (mouse line developed by Bowermann et al. Neuromusc Disord 2012 March;22(3):263-76).

Smn^{2B/-} mice were treated with viral particles at birth (P0) by intracerebroventricular (ICV) injections; ssAAV9-hSM N1 (8×10e¹² vg/kg, 7 μl total volume) was administrated into the right lateral ventricle. Control Smn^{2B/+} littermates and wild-type mice received 7 μl of PBS-MK (1mM MgCl₂, 2.5 mM KCl) at birth using the same procedure. In vivo protocols were designed to assess the lifespan of mutant mice after treatment compared to controls. A group of animals (serie 3, n=10 mice per group) was used to analyze the life expectancy of treated Smn2B/− mice compared to uninjected mutant mice.

Non-treated Smn^{2B/-} mice had a median lifespan at around 26 days of age (n=20). On the contrary, the injection of ssAAV-hSMN1 vectors was able to prolong the lifespan of Smn^{2B/-} mice with differences in the median lifespan (n=10 for each group):
ssAAV9 7210: 228 days
ssAAV9 7209: 335 days
ssAAV 7212: undefined because more than 50% of mice are still alive at 575 days
ssAAVrh10 7210: 209 days
ssAAV9 7211: 103 days.

At day 575, 70% of the ssAAV9-7212 treated mice (n=10) were still alive, showing the impressive survival improvement obtained thanks to the rAAV vector of the present invention.

FIG. 2 show that body weight of mice treated with the vector of the invention is highly improved as compared to untreated mice.

In addition, serial coronal cryostat (16 μm-thicks) sections were collected by a cryostat and processed for anti-ChAT (Cholin Acetyl Transferase) staining. Bilateral counts were performed along the lumbar segment: only large cell bodies in laminae 8 and 9 (ventral horn) of the spinal cord that exhibited ChAT+signal were considered motorneurons.

| | Moy ChAT⁺ MNs |
|---|---|
| Smn+/+ | 11.21 |
| Smn2B/− | 1.85 |
| 7212-injected Smn2B/− | 10.53 |

In conclusion, the expression cassette of the invention provides with a clear prolongation of lifespan after treatment as compared to other expression cassettes including regulatory elements which were reported to be particularly efficient for the expression of a transgene. This result was totally unexpected from the prior publications available with respect to these regulatory elements.

### Example 2

Smn^{2B/-} mice develop a severe phenotype with body weight loss and clinical signs of the disease at around 15 days of age; the current median survival of Smn^{2B/-} mice in our colony is 26 days (mouse line developed by Bowermann et al. Neuromusc Disord 2012 March;22(3):263-76). Smn^{2B/-} mice were treated with viral particles at birth (P0) by intracerebroventricular (ICV) injections into the right lateral ventricle (7 μl total volume). In vivo protocols were designed to assess the lifespan of mutant Smn^{2B/-} mice after treatment (n=10 mice per group) compared to uninjected mutant mice.

To determine the minimal effective dose for increased survival using a single ICV injection of ssAAV9 7212, we tested three doses:
2e12 VG/Kg (low dose)
8e12 VG/Kg (mid dose)
3e13 VG/Kg (high dose)

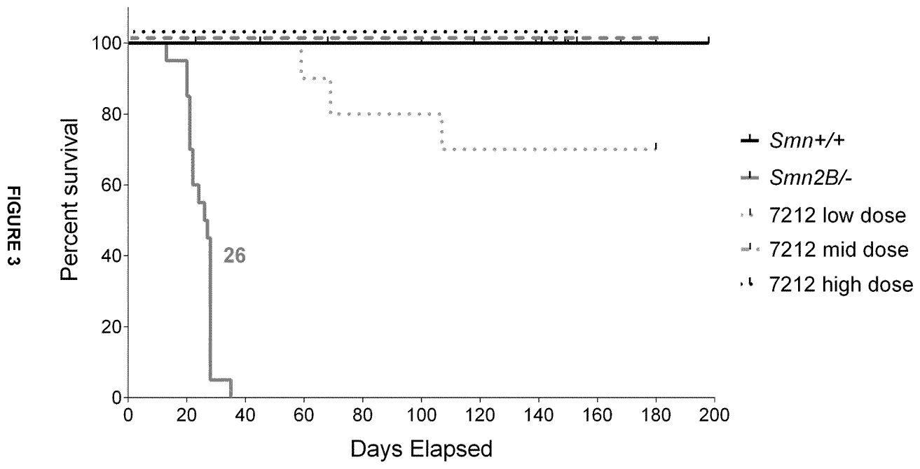

FIG. 3 shows the survival rate of treated and untreated Smn^{2B/-} mice and wild-type animals, with a clear prolongation of lifespan after treatment. At the time of data collection, we were able to calculate the median survival only for not treated Smn^{2B/-} mice (26 days) because more than 50% of ssAAV9-treated Smn^{2B/-} mice were still alive at 155-180 days post-injection.

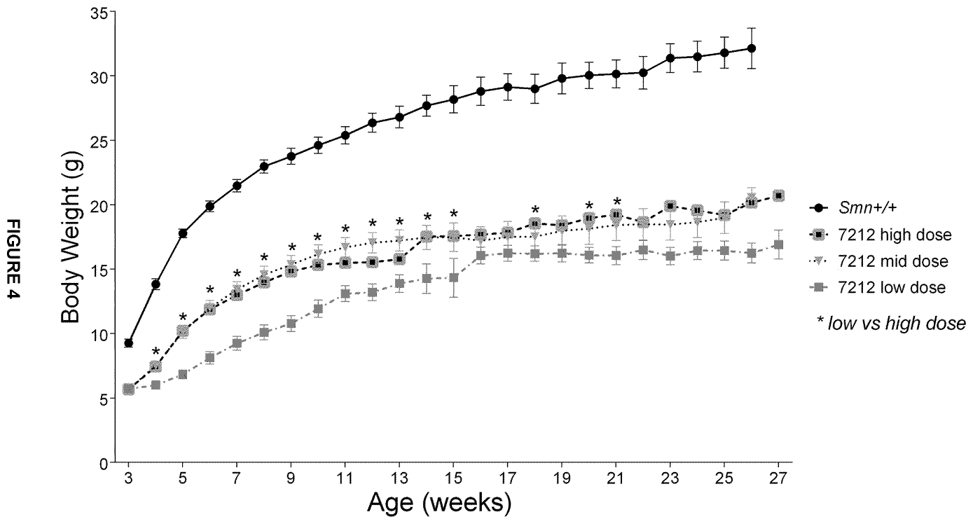

FIG. 4 shows the increase of body weight of treated Smn^{2B/-} mice and wild-type animals, with a weight gain that in part correlates with the dose injected (Multiple T-Test; Error bars=SEM; 14<N<24 per group).

### Conclusion

In order to determine the minimally effective dose of ssAAV9- 7212 after a single ICV injection at birth (P0), we performed a dose response study of the vector. Survival and weight gain was monitored twice per week and compared to the median survival of non-treated Smn^{2B/-} mice. We show that all the doses tested increase the survival rate and confirm the efficacy to rescue the SMA phenotype.

1-14 (canceled)

15. An expression cassette comprising:
a promoter that is the PGK promoter consisting of the sequence shown in SEQ ID NO:1, or a functional variant of said promoter having a nucleotide sequence that is at least 80% identical to SEQ ID NO:1;
a modified intron 2/exon 3 sequence from the human f3 globin gene consisting of the sequence shown in SEQ ID NO:12, or a functional variant having a nucleotide sequence that is at least 80% identical to SEQ ID NO:12;
a polynucleotide sequence encoding a survival of motor neuron (SMN) protein; and
a polyadenylation signal consisting of the sequence shown in SEQ ID NO:7 or SEQ ID NO:8, or a functional variant thereof having a nucleotide sequence that is at least 80% identical to SEQ ID NO:7 or SEQ ID NO:8.

16. The expression cassette according to claim 15, wherein the transgene is the human SMN1 gene.

17. The expression cassette according to claim 15, wherein the polyadenylation signal is selected from group consisting of the SMN1 gene polyadenylation signal, the HBB polyadenylation signal, the bovine growth hormone polyadenylation signal, the SV40 polyadenylation signal, and a synthetic polyA.

18. The expression cassette according to claim 15, wherein said expression cassette has a sequence comprising or consisting of the sequence shown in SEQ ID NO:11, or a sequence that is at least 80% identical to SEQ ID NO:11.

19. A recombinant vector comprising the expression cassette according to claim 15.

20. The recombinant vector according to claim 19, which is a plasmid vector or a viral vector.

21. The recombinant vector according to claim 19, wherein said vector is a recombinant adeno-associated virus (rAAV) vector.

22. The recombinant vector according to claim 21, wherein said rAAV vector has an AAV9 or AAVrh10 capsid.

23. The recombinant vector according to claim 21, wherein said rAAV vector has a single-stranded genome.

24. The recombinant vector according to claim 21, wherein the genome of the rAAV vector is a single-stranded genome which comprises:
an AAV 5′-ITR;
a promoter that is the PGK promoter consisting of the sequence shown in SEQ ID NO:1, or a functional variant of said promoter having a nucleotide sequence that is at least 80% identical to SEQ ID NO:1;
a modified intron 2/exon 3 sequence from the human f3 globin gene consisting of the sequence shown in SEQ ID NO:12, or a functional variant having a nucleotide sequence that is at least 80% identical to SEQ ID NO:12;
a polynucleotide sequence encoding a survival of motor neuron (SMN) protein;
a polyadenylation signal consisting of the sequence shown in SEQ ID NO:7 or SEQ ID NO:8, or a functional variant thereof having a nucleotide sequence that is at least 80% identical to SEQ ID NO:7 or SEQ ID NO:8; and
an AAV 3′-ITR.

25. The recombinant vector according to claim 21, wherein the genome of the rAAV vector is a single-stranded genome which comprises:
an AAV 5′-ITR;
an expression cassette having a sequence comprising or consisting of the sequence shown in SEQ ID NO:11, or a sequence that is at least 80% identical to SEQ ID NO:11;
an AAV 3′-ITR.

26. The recombinant vector according to claim 21, wherein the genome of the rAAV vector comprises AAV2 inverted terminal repeats.

27. A method of treating spinal muscular atrophy comprising the administration of an expression cassette according to claim 15, or a recombinant vector comprising said expression cassette to a subject in need of treatment.


SEQUENCE LISTING (12 sequences)

SEQ ID NO: 1 - DNA, 507 bp, artificial
  human PGK promoter
ttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 2 - DNA, 885 bp, artificial
  hSMN1 ORF
atggcgatgagcagcggcggcagtggtggcggcgtcccggagcaggaggattccgtgctgttccggcgcggcacaggccagagcgatgattctgacatttgggatgatacagcactgataaaagcatatgataaagctgtggcttcatttaagcatgctctaaagaatggtgacatttgtgaaacttcgggtaaaccaaaaaccacacctaaaagaaaacctgctaagaagaataaaagccaaaagaagaatactgcagcttccttacaacagtggaaagttggggacaaatgttctgccatttggtcagaagacggttgcatttacccagctaccattgcttcaattgattttaagagagaaacctgtgttgtggtttacactggatatggaaatagagaggagcaaaatctgtccgatctactttccccaatctgtgaagtagctaataatatagaacaaaatgctcaagagaatgaaaatgaaagccaagtttcaacagatgaaagtgagaactccaggtctcctggaaataaatcagataacatcaagcccaaatctgctccatggaactcttttctccctccaccaccccccatgccagggccaagactgggaccaggaaagccaggtctaaaattcaatggcccaccaccgccaccgccaccaccaccaccccacttactatcatgctggctgcctccatttccttctggaccaccaataattcccccaccacctcccatatgtccagattctcttgatgatgctgatgctttgggaagtatgttaatttcatggtacatgagtggctatcatactggctattatatgggtttcagacaaaatcaaaaagaaggaaggtgctcacattccttaaattaa

SEQ ID NO: 3 - DNA, 885 bp, artificial
  hSMN1co_ATUM
atggccatgagcagcggtggttcaggcggtggagtgcctgagcaagaggattcggtgctgttcaggaggggcaccggacagtccgacgactccgatatttgggatgataccgcactgattaaggcatacgacaaggccgtggcgtccttcaagcacgcgctgaagaatggcgacatctgcgaaacctcaggaaagcccaagactaccccgaagcgcaaaccggccaagaagaacaagtcgcagaagaagaacactgccgccagcctccaacagtggaaagtcggggacaagtgctccgccatctggtccgaggacggatgtatctacccggccaccattgcctccatcgacttcaagcgcgagacttgcgtggtcgtgtataccggatacggcaaccgcgaagaacagaatctcagcgatctgctgtcacccatctgcgaagtggcgaacaacatcgaacagaacgcccaggagaacgaaaacgagtcccaagtctccaccgacgaatccgagaactcgagatcacccgggaacaagtccgacaacattaagccgaagtctgccccctggaactccttccttccgcctccgccacctatgcccggacccagacttgggccggggaaacctggtctgaagttcaatggaccacctccgcctcctccacctcctcccccacacctcctgtcctgctggttgcccccgtttccctccggaccgcctattatcccaccaccgcctcctatctgcccggactccctggacgatgccgacgctctggggagcatgctgatctcgtggtacatgagcggataccacaccggctactacatgggattccggcagaaccagaaggaaggccggtgttcgcattcgctgaactga

SEQ ID NO: 4 - DNA, 885 bp, artificial
  hSMN1co_Genc3
atggcaatgagcagcggaggaagcggaggaggagttcctgaacaggaggacagcgtgctgttcaggagaggaaccggacagagcgacgacagtgacatctgggacgacaccgcactgatcaaagcctacgacaaggcagtggcaagctttaagcacgccctgaagaacggagatatttgtgagacaagcggcaagcccaaaaccacacccaaacgcaagcccgctaagaaaaacaagtcacagaagaagaacacagctgcctcactgcagcaatggaaggtgggagacaagtgcagcgcaatctggagcgaggacggatgtatctaccccgcaacaatagccagcatcgacttcaagagagaaacctgcgtggtggtgtacaccggctacggaaacagagaagagcagaacctgagcgacctgctgagccctatatgcgaggtggctaataacatcgagcaaaacgcccaggagaacgagaacgagagccaggttagcaccgatgagagcgaaaacagcagaagccccggcaacaaaagcgacaacatcaagcccaagagcgccccatggaacagcttcctgcctcctcctccacctatgcctggacctagactgggaccaggaaaacccggactgaaattcaacgggccaccccctccaccaccacctcctcctcctcatctgctgtcatgctggctccctcctttcccttccggacctcctatcatcccccctcctcctcctatctgccctgattctctcgacgacgccgacgctctgggatctatgctgatctcctggtacatgtccggctaccacaccggttactacatgggcttcagacagaaccaaaaggaaggccggtgttcccacagcctgaactga

SEQ ID NO: 5 - DNA, 885 bp, artificial
  hSMN1co_Genw4
atggccatgtcctccggaggaagcggaggaggcgtgcctgaacaggaggacagcgtgctgtttaggaggggcacaggccagagcgacgactccgacatctgggatgacaccgctctgatcaaggcctacgacaaggccgtggccagcttcaagcacgctctgaagaacggcgacatctgtgagacctccggcaagcccaagaccacacccaagaggaagcccgccaagaagaacaagtcccagaagaagaacaccgccgcttccctgcagcagtggaaggtgggcgacaagtgctccgctatctggtccgaggatggctgcatctaccccgccaccattgcctccatcgacttcaagagggagacctgcgtggtggtgtacaccggctacggcaacagggaggagcagaacctgagcgacctgctgagccctatctgcgaggtggctaacaacatcgagcagaacgcccaagagaatgagaacgagtcccaggtgagcacagacgagagcgagaattccaggtcccccggcaataagagcgacaacatcaagcccaagagcgccccctggaacagctttctgcctcctcccccccctatgcctggccctagactcggacccggaaaacccggcctgaagttcaacggacctccccctcctcctcctcctcctcctccccatctgctgagctgctggctgcccccttttccctccggacctcccatcattcctcctcctccccccatttgccccgactccctggacgatgccgacgctctgggctccatgctgatcagctggtacatgtccggctaccacaccggctactacatgggcttcaggcagaaccagaaagagggcaggtgctcccactccctgaactga

SEQ ID NO: 6 - DNA, 885 bp, artificial
  hSMN1co_MMV3.5
atggccatgagctctggagggtctggaggaggagtgcctgagcaggaggactctgtgctgttcagaagaggcacaggccagtctgatgattctgacatctgggatgacacagccctgatcaaggcctatgacaaggctgtggcttccttcaagcatgccctgaagaacggagacatctgtgagacttctggcaagccaaagaccacacccaagagaaagcctgccaagaagaacaagagccagaagaagaacactgctgccagcctgcagcagtggaaggtgggggacaagtgctctgctatctggtcagaggatggctgtatctaccctgccaccattgccagcattgacttcaagagagagacctgtgtggtggtgtacacaggctatggcaacagagaggagcagaacctgtctgacctgctgagccccatctgtgaggtggccaacaacattgagcagaatgcccaggagaatgagaatgagagccaggtgagcacagatgagtctgagaacagcagatctcctggcaacaagtctgacaatatcaagcccaagtctgccccctggaacagcttcctgccccctcctcctcctatgcctggccccagactgggacctggcaagcctggcctgaagttcaacggcccccctccccctccccctccccctccccctcacctgctgagctgctggctgccccccttcccctctggcccccccatcatcccccctcctccccctatctgccctgactctctggatgatgctgatgccctgggcagcatgctgatcagctggtatatgtctggctaccacacaggctactacatgggcttcagacagaaccagaaggagggcagatgcagccacagcctgaactga

SEQ ID NO: 7 - DNA, 766 bp, artificial
  Human beta globin gene polyadenylation signal
attcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatc

SEQ ID NO: 8 - DNA, 574 bp, artificial
  Human beta globin gene polyadenylation signal - part
aaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatc

SEQ ID NO: 9 - DNA, 6 bp, artificial
  Kozak sequence
gccacc

SEQ ID NO: 10 - DNA, 49 bp, artificial
  synthetic polyA
aataaaagatctttattttcattagatctgtgtgttggttttttgtgtg

SEQ ID NO: 11 - DNA, 2610 bp, artificial
  7212 SMN expression cassette
ttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcctgttcatacctcttatcttcctcccacagctcctgggcaacgtgctggtctgtgtgctggcccatcactttggcaaagaattcgccaccatggcgatgagcagcggcggcagtggtggcggcgtcccggagcaggaggattccgtgctgttccggcgcggcacaggccagagcgatgattctgacatttgggatgatacagcactgataaaagcatatgataaagctgtggcttcatttaagcatgctctaaagaatggtgacatttgtgaaacttcgggtaaaccaaaaaccacacctaaaagaaaacctgctaagaagaataaaagccaaaagaagaatactgcagcttccttacaacagtggaaagttggggacaaatgttctgccatttggtcagaagacggttgcatttacccagctaccattgcttcaattgattttaagagagaaacctgtgttgtggtttacactggatatggaaatagagaggagcaaaatctgtccgatctactttccccaatctgtgaagtagctaataatatagaacaaaatgctcaagagaatgaaaatgaaagccaagtttcaacagatgaaagtgagaactccaggtctcctggaaataaatcagataacatcaagcccaaatctgctccatggaactcttttctccctccaccaccccccatgccagggccaagactgggaccaggaaagccaggtctaaaattcaatggcccaccaccgccaccgccaccaccaccaccccacttactatcatgctggctgcctccatttccttctggaccaccaataattcccccaccacctcccatatgtccagattctcttgatgatgctgatgctttgggaagtatgttaatttcatggtacatgagtggctatcatactggctattatatgggtttcagacaaaatcaaaaagaaggaaggtgctcacattccttaaattaaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatc

SEQ ID NO: 12 - DNA, 446 bp, artificial
  modified intron 2/exon 3 sequence from the human beta-globin gene
gtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcctgttcatacctcttatcttcctcccacagctcctgggcaacgtgctggtctgtgtgctggcccatcactttggcaaagaattc